(19) United States
(12) Patent Application Publication
(10) Pub. No.: US 2025/0127453 A1
(43) Pub. Date: Apr. 24, 2025

(54) PREDICTION METHOD FOR BREAKTHROUGH PAIN OF SUBJECT AND ANALYSIS APPARATUS

(71) Applicant: SAMSUNG LIFE PUBLIC WELFARE FOUNDATION, Seoul (KR)

(72) Inventors: Yeong Hak BANG, Seoul (KR); Yoon Ho CHOI, Seoul (KR); Seok Jin KIM, Seoul (KR)

(21) Appl. No.: 18/396,144

(22) Filed: Dec. 26, 2023

(30) Foreign Application Priority Data
Oct. 20, 2023 (KR) 10-2023-0141443

(51) Int. Cl. A61B 5/00; G16H 50/20

(52) U.S. Cl. CPC A61B 5/4824; G16H 50/20

(57) ABSTRACT

Proposed is a prediction method for breakthrough pain of a subject, the method including receiving pain score data collected from a subject for a predetermined time by means of an analysis apparatus, preprocessing the pain score data by means of the analysis apparatus, and inputting the preprocessed pain score data into a deep learning model trained in advance and predicting whether breakthrough pain will be generated in the subject in a point in time in the future on the basis of a prediction value output from the deep learning model by means of the analysis apparatus.

### CROSS REFERENCE TO RELATED APPLICATION

This application claims the benefit under 35 U.S.C. § 119 (a) of Korean Patent Applications No. KR 10-2023-0141443, filed Oct. 20, 2023, in the Korean Intellectual Property Office, the entire disclosure of which is incorporated herein by reference for all purposes.

### FIELD

The following description relates to a technique of predicting breakthrough pain of cancer patients using artificial intelligence.

### BACKGROUND

Breakthrough pain is a phenomenon that pain becomes transiently and rapidly worse in cancer patients. Breakthrough pain results in deterioration of the quality of life of cancer patients. At present, pain is controlled by combinations of long-acting analgesics and fast-acting analgesics at the clinical sites. However, even though fast-acting analgesics are used, cancer patients unavoidably suffer from breakthrough pain until the effect of a medicine is generated after medication.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided a prediction method for breakthrough pain of a subject includes: receiving pain score data collected from a subject for a predetermined time by means of an analysis apparatus; preprocessing the pain score data by means of the analysis apparatus; and inputting the preprocessed pain score data into a deep learning model trained in advance and predicting whether breakthrough pain will be generated in the subject in a point in time in the future on the basis of a prediction value output from the deep learning model by means of the analysis apparatus.

In another aspect, there is provided an analysis apparatus for predicting breakthrough pain of a subject includes: an interface device configured to receive pain score data collected from a subject for a predetermined time; a storage device configured to storing a deep learning model configured to predict whether breakthrough pain will be generated by receiving pain information of a patient; and an computing device configured to preprocess the received pain score data and predict whether breakthrough pain of the patient will be generated at a point in time in the future on the basis of a prediction value that is output by inputting the preprocessed pain score data into a deep learning model trained in advance.

### BRIEF DESCRIPTION OF THE DRAWINGS

FIG. 1 illustrates an example of a system that predicts breakthrough pain using information of cancer patients.

FIG. 2 illustrates an example of a learning process of a corresponding deep learning model.

FIGS. 3A, 3B, 3C, and 3D illustrate an example of a process of preprocessing pain data.

FIG. 4 illustrates an example of a deep learning model that predicts breakthrough pain at a point in time in the future by receiving preprocessed pain information of patients.

FIG. 5 illustrates an example of a process of verifying a deep learning model that predicts breakthrough pain.

FIGS. 6A, 6B, and 6C are AUC curves of an LSTM-based prediction model. and

FIG. 7 illustrates an example of an analysis apparatus for predicting breakthrough pain.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

As used herein, the term “and/or” includes any one and any combination of any two or more of the associated listed items.

The terminology used herein is for describing various examples only, and is not to be used to limit the disclosure. The articles “a,” “an,” and “the” are intended to include the plural forms as well, unless the context clearly indicates otherwise. The terms “comprises,” “includes,” and “has” specify the presence of stated features, numbers, operations, members, elements, and/or combinations thereof, but do not preclude the presence or addition of one or more other features, numbers, operations, members, elements, and/or combinations thereof.

The technology to be described hereafter is a technique that predicts breakthrough pain of cancer patients. Further, the technology to be described may be used to predict breakthrough pain of patients with specific types of diseases regardless of the types of diseases.

The technology to be described predicts breakthrough pain using time-series data that can be collected from cancer patients. The time-series data may include pain scores evaluated over time and other clinical information. The other clinical information may include age, sex, disease type, other Electronic Medical Record (EMR) data.

It is assumed hereafter that an analysis apparatus performs breakthrough pain prediction on the basis of time-series data of cancer patients using a learning model. The analysis apparatus may be implemented into various devices that can process data and control the operation of a learning model. For example, the analysis apparatus may be a PC, a server on a network, a smart device, a chipset embedded with an exclusive program, etc.

Any one of various types of models may be used as the learning model. For example, as the learning model, there are a decision tree, a random forest, a K-nearest neighbor (KNN), Naive Bayes, a support vector machine (SVM), an artificial neural network (ANN), etc. In particular, a deep learning model that can process time-series data can be used as the learning model.

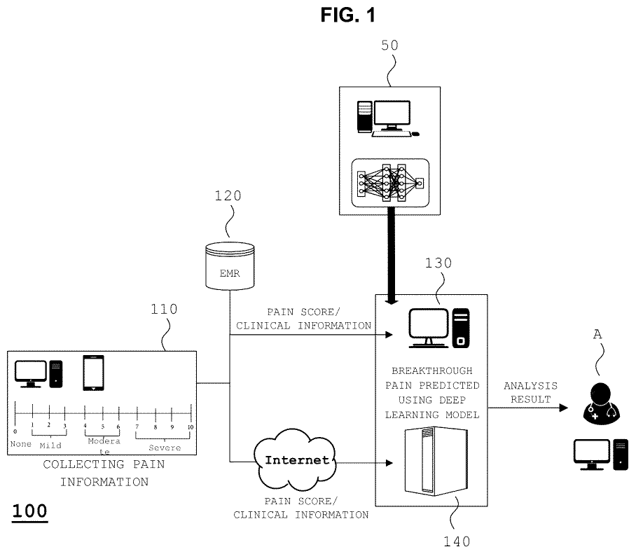

FIG. 1 illustrates an example of a system 100 that predicts breakthrough pain using information of cancer patients. In FIG. 1, a computer terminal 130 and a server 140 are exemplified as analysis apparatuses.

A user terminal 110 collects a pain score (NRS) of a subject (patient). The NRS score is in the range of [0, 10]. The NRS score is classified into mild pain, moderate pain, severe pain, depending on the rate. A pain score may be information that is calculated by evaluating the state of a subject over time. The user terminal 100 can store a collected pain score (NRS) into an Electronic Medical Record (EMR) 120 or a separate database (DB).

A learning device 50 constructs a deep learning model that predicts breakthrough pain by analyzing patient information. The learning device 50 means a computing device that can preprocess patient data and perform learning through a deep learning model. The learning device 50 can construct a deep learning model using predetermined learning data. The learning process or the model structure will be described below. The computer terminal 130 and the server 140 are used to obtain a constructed deep learning model and analyze a subject.

In FIG. 1, a user A can analyze the pain score and clinical information of a patient using the computer terminal 130. The computer terminal 130 can receive the pain score and the clinical information of a patient from the user terminal 110 or the EMR 120 through a wire or wireless network. Depending on cases, the computer terminal 130 may be a device physically connected with the user terminal 110. The computer terminal 130 can uniformly preprocess the pain score and/or the clinical information of a patient. The computer terminal 130 can predict breakthrough pain on the basis of a value that is output from a deep learning model that receives the pain score and the clinical information of a patient. The user A can check the analysis result through the computer terminal 130.

The server 140 can receive the pain score and clinical information of a patient from the user terminal 110 or the EMR 120 through a wireless network. The server 140 can uniformly preprocess the pain score and/or the clinical information of a patient. The server 140 can predict breakthrough pain on the basis of a value that is output from a deep learning model that receives the pain score and the clinical information of a patient. The server 140 can transmit the analysis result to the terminal of the user A.

The computer terminal 130 and/or the server 140 may store the analysis result in the EMR 120.

Hereafter, a deep learning model that predicts breakthrough pain of cancer patients and the learning process of the deep learning model are described.

Researchers used a dataset of 3,431 persons excluding persons 2,697 persons who had a surgical operation 28,173 and persons with a non-zero numerical rating scale (NRS) score less than 20 from 34,301 patients who visited the Hematology & Oncology department of Samsung Medical Center, which is an affiliated research institute, in the period of July 2016˜ February, 2020.

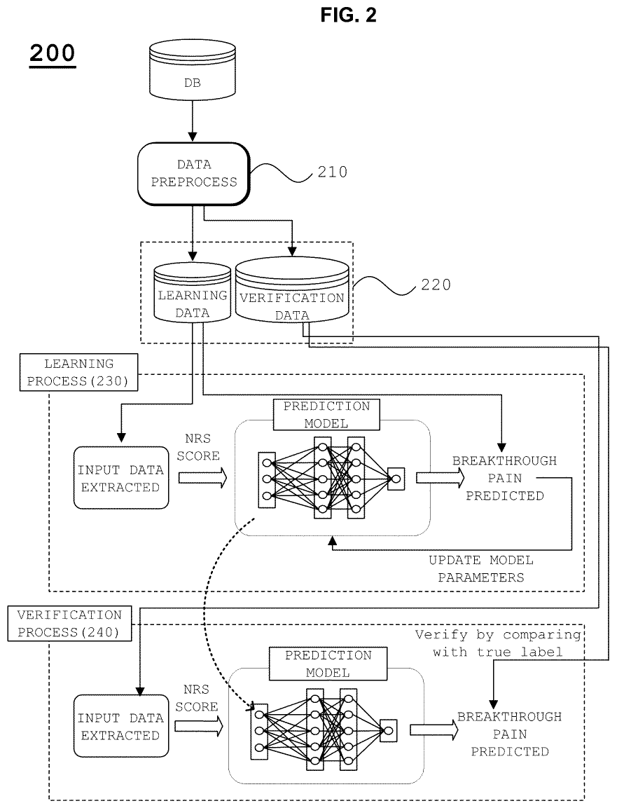

FIG. 2 illustrates an example of a learning process 200 of a corresponding deep learning model. The deep learning model is a model that predicts (classifies) breakthrough pain using patient information. Accordingly, the deep learning model may also be called a prediction model.

A database (DB) can store the pain scores and clinical information of patients included in the dataset. The pain scores are time-series data that are collected for predetermined time. The medical staff collected pain scores with predetermined time intervals in accordance with an NRS scale. The medical staff collected pain scores everyday at 05:00, 13:00, and 21:00. Further, the medical staff collected corresponding information also when breakthrough pain is generated in patients even through it is another time. The clinical information may include age, sex, and other items. The researchers defined NRS score 4, which is the reference of medication of narcotic analgesics, as breakthrough pain of cancer patient in accordance with a guideline. In this case, the case over NRS score 4 corresponds to breakthrough pain.

The learning device can uniformly preprocess raw data of patients (210).

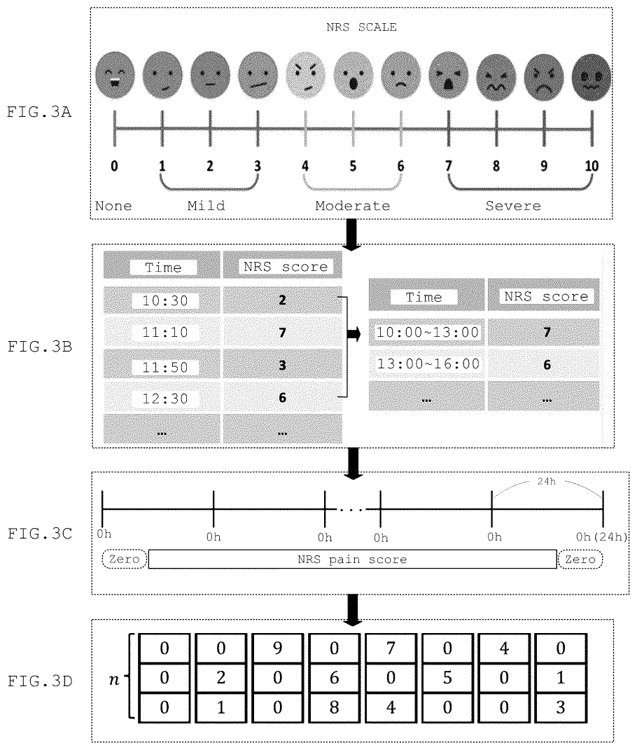

FIGS. 3A to 3D illustrate an example of a process of preprocessing pain data.

FIG. 3A is an example of the NRS scale with pain score.

The learning device divides pain scores that are time-series information into predetermined time sections. The learning device assigns a pain score to each section in accordance with several time sections (bins). When several pain scores exist in one section, the learning device can set the highest score as the score of the section. One bin may be defined as a predetermined time length (e.g., τ hours). FIG. 3B is an example of dividing pain scores into predetermined time sections (τ=3 hours).

Breakthrough pain may be evaluated differently even in one patient or different patients. For example, breakthrough pain may have a specific pattern for each patient in accordance with the medical state of the patients. Breakthrough pain may be evaluated differently for patients in accordance with management and the state of the patients. The learning device can process the entire data in the unit of predetermined time sections. The researchers processed pain score data in the unit of 24 hours (00:00˜ 24:00). Further, the learning device can perform zero padding on parts that are not in the original data to set the start and the end of the pain score data as 00:00. FIG. 3C is an example of a process of processing pain score data, which were continuously collected on an n day, in the unit of 24 hours.

It was described with reference to FIGS. 3B to 3C that pain score data are divided into predetermined time sections (τ=3 hours) and then the data are divided in the unit of day, but the order of this process may be reversed.

The learning device can convert the pain score data on the n day divided into bins of τ time length into a 1×((24/τ)) vector type into a (24/τ)×n matrix type. FIG. 3D is an example in which pain score data processed in the unit of 24 hours were converted into a matrix type. In FIG. 3D, one row is an example of pain score data in corresponding time sections for one day and the columns are examples of pain score data collected in the unit of n days.

Further, as input data, clinical information such as sex and age of corresponding patients may be used other than the pain score.

Thereafter, the learning device can construct a prediction model using the pain score data preprocessed into the matrix type. Further, the learning device may construct a prediction model further using clinical information together with the pain score data that are time-series data.

The researchers constructed different types of deep learning models. The deep learning models include a recurrent neural network (RNN), a long short term memory (LSTM), a gated recurrent unit (GRU), a bidirectional long short-term memory (Bi-LSTM), a hybrid of the convolutional neural network, long short-term memory), and a transformer. Further, the researchers evaluated the constructed models while variously changing the time section lengths of data. Further, the researchers constructed the models in time section period units, τ∈{1, 2, 3, 4, 6, 8, 12}.

The learning device divides the collected entire data into learning data and verification data (220). The researchers used the data of 2,745 persons (80%) of a dataset of 3.431 persons as learning data and used the data of 686 persons (20%) as test data.

The learning device trains the prediction model using learning data (220). The learning device performs the learning process by extract one input data from the learning data. The learning device updates the parameters of the prediction model by comparing a value (breakthrough pain prediction value) output from the prediction model receiving the input data with a value of true label. The learning device repeats the learning process using the data pertaining to the learning data.

When finishing the learning process, the learning device verifies the trained prediction model (230). The learning device performs a verification process by extracting one of verification data. The learning device outputs a breakthrough pain prediction value by inputting the selected data into the prediction model and performs verification by comparing the predicted result with a value of true label.

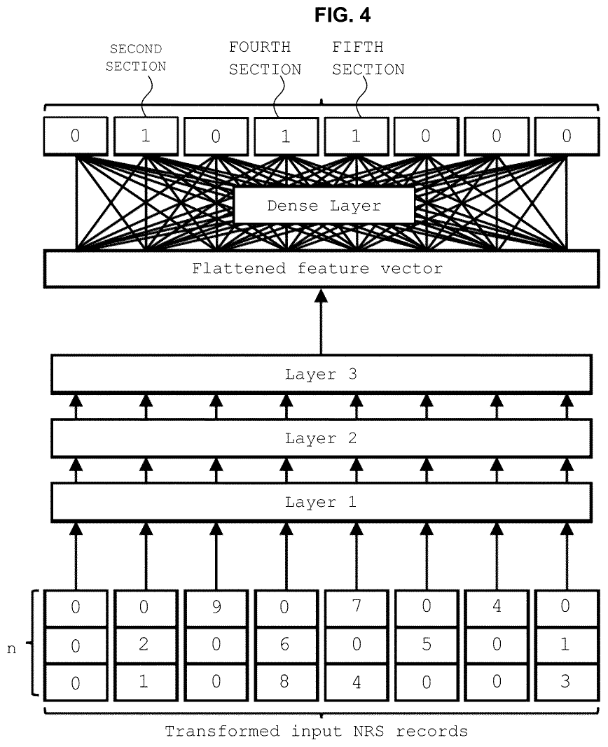

FIG. 4 is an example of a deep learning model that predicts breakthrough pain at a point in time in the future by receiving preprocessed pain information of patients. An analysis apparatus receives pain score data at a point in time in the past of a patient who is a prediction subject. The analysis apparatus uniformly preprocesses the pain score data. The preprocessing is the same as the above description. The analysis apparatus can convert the pain score data on an n day divided into bins of τ time length into a (24/τ)×n matrix type. FIG. 4 is an example when τ time=3 hours. The analysis apparatus inputs the pain data of a matrix type into a constructed deep learning model. The deep learning model may be composed of many convolution layers (e.g., Layer 1˜ Layer 3) and a dense layer. The dense layer can finally output information of predicting the possibility of generation of breakthrough pain for each time section (bin) using an activation function. The analysis apparatus can predict whether breakthrough pain will be generated for 24 hours from the last point in time at which the information of a patient was collected, on the basis of information that is output from the deep learning model. For example, the analysis apparatus can predict that there is a high possibility of generation of breakthrough pain in the second section (03:00˜ 06:00), the fourth section (09:00˜ 12:00), and the fifth section (12:00˜ 15:00) in FIG. 4.

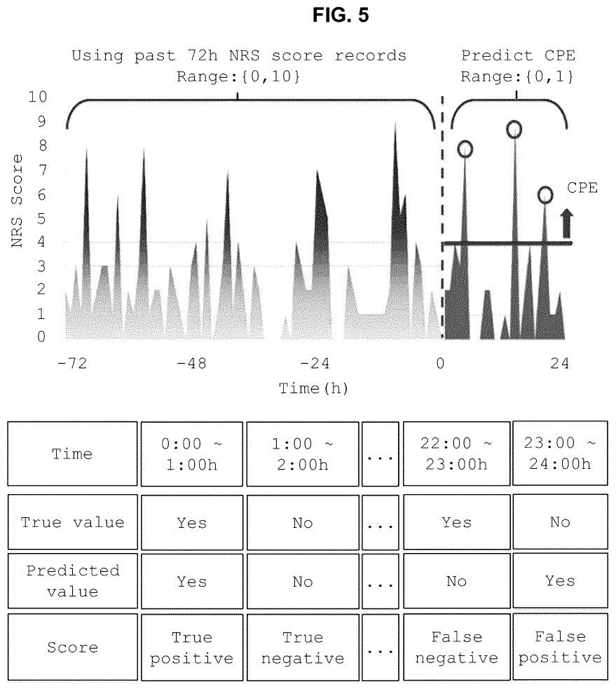

FIG. 5 is an example of a process of verification a deep learning model that predicts breakthrough pain. The analysis apparatus predicts breakthrough pain for 24 hours immediately after using pain score data of 72 hours. In this case, the analysis apparatus can predict the possibility of generation of breakthrough pain on the basis of pain scores that the deep learning model predicts in accordance with time sections. For example, when the pain score predicted in a specific time section is 4 or more, the analysis apparatus can determine generation of breakthrough pain (1 in a binary value) in the section, and when the pain score predicted in a specific time section is less than 4, the analysis apparatus can determine non-generation of breakthrough pain (0 in a binary value) in the section.

The analysis apparatus can verify the performance of the model by comparing a breakthrough pain prediction value from the deep learning model predicted in a time section with a value of true label. A Matthews correlation coefficient (MCC) may be used as an verification index. The MCC is a value in the range of [−1, 1], and when the MCC is closer to +1, it means higher accuracy. The following table 1 shows the performance of models. In the table 1, input data are classified into data for the past 24 hours, data for the past 72 hours, and data for the past 120 hours. Further, the data of which the time sections are different as τ∈{1, 2, 3, 4, 6, 8, 12} were used as the input data.

TABLE 1

| Input | | Time-bin length τ | | | | | | |
|---|---|---|---|---|---|---|---|---|
| length | Model | 1 | 2 | 3 | 4 | 6 | 8 | 12 |
| 24 | Transformer | 0.1616 | 0.2319 | 0.2813 | 0.3279 | 0.3735 | 0.4059 | 0.4174 |
| | CNN + LSTM | 0.1595 | 0.2265 | 0.2762 | 0.2988 | 0.3708 | 0.3791 | 0.4178 |
| | Bi-LSTM | 0.1666 | 0.2330 | 0.2807 | 0.3325 | 0.3744 | 0.4062 | 0.4180 |
| | LSTM | 0.1721 | 0.2417 | 0.2900 | 0.3365 | 0.3718 | 0.4074 | 0.4179 |
| | GRU | 0.1696 | 0.2406 | 0.2912 | 0.3351 | 0.3705 | 0.4055 | 0.4171 |
| | RNN | 0.1669 | 0.2328 | 0.2832 | 0.3320 | 0.3703 | 0.4084 | 0.4167 |
| 72 | Transformer | 0.1764 | 0.2598 | 0.3116 | 0.3589 | 0.4100 | 0.4476 | 0.4685 |
| | CNN + LSTM | 0.1872 | 0.2562 | 0.3116 | 0.3617 | 0.4118 | 0.4529 | 0.4485 |
| | Bi-LSTM | 0.1805 | 0.2501 | 0.3016 | 0.3514 | 0.4079 | 0.4523 | 0.4745 |
| | LSTM | 0.1889 | 0.2650 | 0.3238 | 0.3722 | 0.4233 | 0.4624 | 0.4712 |
| | GRU | 0.1938 | 0.2735 | 0.3170 | 0.3710 | 0.4223 | 0.4618 | 0.4796 |
| | RNN | 0.1829 | 0.2537 | 0.3149 | 0.3636 | 0.4136 | 0.4478 | 0.4717 |
| 120 | Transformer | 0.1761 | 0.2623 | 0.3213 | 0.3688 | 0.4315 | 0.4620 | 0.4879 |
| | CNN + LSTM | 0.1929 | 0.2658 | 0.3207 | 0.3717 | 0.4218 | 0.4615 | 0.9000 |
| | Bi-LSTM | 0.1815 | 0.2496 | 0.3068 | 0.3581 | 0.4142 | 0.4680 | 0.4830 |
| | LSTM | 0.1861 | 0.2635 | 0.3231 | 0.3771 | 0.4278 | 0.4730 | 0.4927 |
| | GRU | 0.1963 | 0.2717 | 0.3196 | 0.3791 | 0.4267 | 0.4848 | 0.4923 |
| | RNN | 0.1788 | 0.2537 | 0.3097 | 0.3734 | 0.4206 | 0.4674 | 0.4924 |

Referring to Table 1, the LSTM-based model showed generally high performance. The LSTM model showed the highest performance as MCC=0.4927 in the example that used a time section of 12 hours for the data of 120 hours.

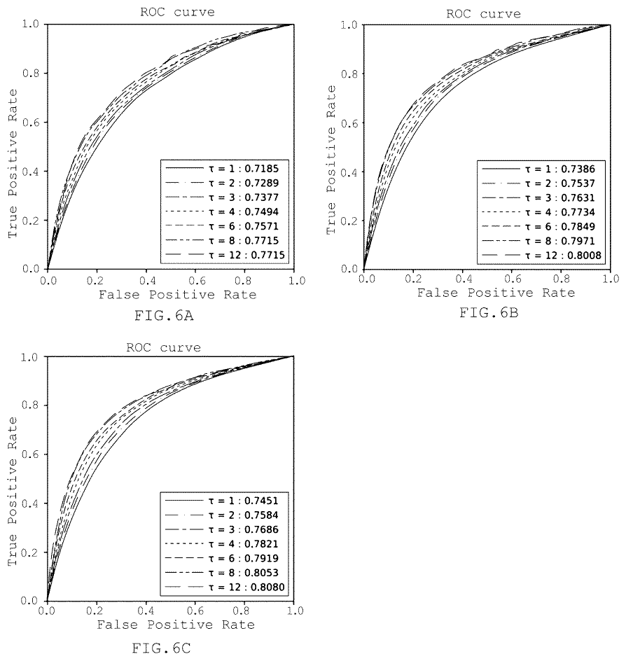

FIGS. 6A to 6C are AUC curves of an LSTM-based prediction model. FIG. 6A is the result when using pain score data for the past 24 hours, FIG. 6B is the result when using pain score data for the past 72 hours, and FIG. 6C is the result when using pain score data for the past 120 hours. The LSTM-based prediction model showed meaningful performance in all cases for the data of various time sections.

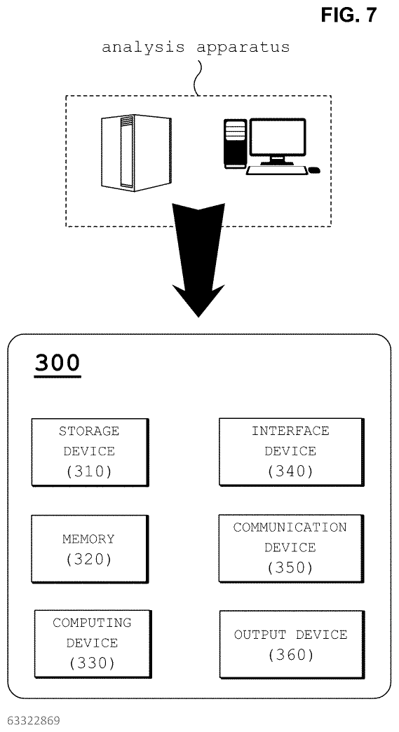

FIG. 7 is an example of an analysis apparatus 300 for predicting breakthrough pain. The analysis apparatus 300 corresponds to the analysis apparatuses (130 and 140 in FIG. 1) described above. The analysis apparatus 300 may be implemented physically in various types. For example, the analysis apparatus 300 may have the types of a computer device such as a PC, the server of a network, an exclusive chipset for data processing, etc.

The analysis apparatus 300 may include a storage device 310, a memory 320, a computing device 330, an interface device 340, a communication device 350, and an output device 360.

The storage device 310 can store past pain score data of patients. The pain score data may be data collected for a predetermined time such as past 24 hours, 72 hours, or 120 hours.

The storage device 310 can designate clinical information (sex, age, etc.) of patients.

The storage device 310 can store a deep learning model that predicts breakthrough pain. In this case, the storage device 310 may store also several models constructed using time data of different lengths.

The storage device 310 can store an analysis result.

The memory 320 can store data, information, etc. that are created when the analysis apparatus 300 predicts breakthrough pain using pain score data of patients.

The interface device 340 is a device that receives predetermined instructions and data that are input from the outside.

The interface device 340 can receive pain score data of subjects from an input device physically connected thereto or an external storage device. Further, the interface device 340 can receive clinical information of subjects from an input device physically connected thereto or an external storage device.

The interface device 340 may transmit the result of predicting breakthrough pain to an external object. The prediction result may be composed of breakthrough pain prediction values for 24 hours from the point in time at which the data of a patient are collected.

The communication device 350 means a configuration that receives and transmits predetermined information through a wire or wireless network.

The communication device 350 can receive pain score data of subjects from an external object. The communication device 350 can receive clinical information of subjects.

Alternatively, the communication device 350 may transmit a prediction result of breakthrough pain to an external object such as a user terminal.

Meanwhile, the interface device 340 is a meaning that includes an interface that transmits data or information received from the communication device 350 into the analysis apparatus 300.

The output device 360 is a device that outputs predetermined information. The output device 360 can output an interface required in a data processing process, an analysis result, etc.

The computing device 330 can uniformly preprocess initial pain score data of subjects. The preprocessing may include a process of creating pain score data of a matrix type through dividing data in accordance with time sections and processing data in the unit of 24 hours, as described above.

The computing device 300 can input (preprocessed) pain score data into a deep learning model trained in advance and can predict whether breakthrough pain of the corresponding subject will be generated on the basis of a probability value that is output from the deep learning model.

In this case, as the deep learning model, a deep learning model suitable for input data can be used in accordance with the time lengths of past data collected from patients and/or the lengths of time section units dividing pain data.

The computing device 330 can determine whether breakthrough will be generated in a specific section of future time sections on the basis of an output value of the deep learning model.

The computing device 330 may be a device such as am arithmetic unit, a processor, an AP, and a program-embedded chip that process data and process predetermined computation.

Further, the method of predicting breakthrough pain of cancer patients described above may be implemented into a program (or an application) including an executable algorithm that can be executed in a computer. The program may be stored and provided in a transitory or non-transitory computer readable medium.

The non-transitory computer readable medium is not a medium that stores data for a short time such as a cache, and a memory, but a medium that can semi-permanently store data and can be read out by a device. In detail, the various applications or programs described above may be stored and provided in a non-transitory readable medium such as a CD, a DVC, a hard disk, a Blu-ray disc, a USB, a memory card, a read-only memory (ROM), a programmable read only memory (PROM), an Erasable PROM (EPROM), an Electrically EPROM (EEPROM), or a flash memory.

The transitory computer readable medium means various RAMs such as a Static RAM (SRAM), a Dynamic RAM (DRAM), a synchronous DRAM (SDRAM), a Double Data Rate SDRAM (DDR SDRAM), an Enhanced SDRAM (ESDRAM), a Synclink DRAM (SLDRAM), and a Direct Rambus RAM (DRRAM).

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A prediction method for breakthrough pain of a subject, the method comprising:
receiving pain score data collected from a subject for a predetermined time by means of an analysis apparatus;
preprocessing the pain score data by means of the analysis apparatus; and
inputting the preprocessed pain score data into a deep learning model trained in advance and predicting whether breakthrough pain will be generated in the subject in a point in time in the future on the basis of a prediction value output from the deep learning model by means of the analysis apparatus.

2. The prediction method of claim 1, wherein the preprocessing comprises:
dividing the pain score data in accordance with a predetermined time length by means of the analysis apparatus; and
dividing the pain score data divided in accordance with the predetermined time lengths in predetermined time section units and setting a pain score for each of the predetermined time section units.

3. The prediction method of claim 2, wherein the deep learning model outputs a probability value of generation of breakthrough pain in a unit of the predetermined time sections.

4. An analysis apparatus comprising:
an interface device configured to receive pain score data collected from a subject for a predetermined time;
a storage device configured to storing a deep learning model configured to predict whether breakthrough pain will be generated by receiving pain information of a patient; and
a computing device configured to preprocess the received pain score data and predict whether breakthrough pain of the patient will be generated at a point in time in the future on the basis of a prediction value that is output by inputting the preprocessed pain score data into a deep learning model trained in advance.

5. The analysis apparatus of claim 4, wherein the computing device divides the pain score data in accordance with a predetermined time length, further divides the predetermined time length into predetermined time section units, and sets a pain score for each of the predetermined time section units.

6. The analysis apparatus of claim 5, wherein the deep learning model outputs a probability value of generation of breakthrough pain in a unit of the predetermined time sections.
